(19) United States
(12) Patent Application Publication
(10) Pub. No.: US 2023/0393050 A1
(43) Pub. Date: Dec. 7, 2023

(54) PARTICLE CLASSIFICATION AND SORTING SYSTEMS AND METHODS

(71) Applicant: Engender Technologies Limited, Hamilton (NZ)

(72) Inventors: Lakshmi KRISHNAN, Auckland (NZ); Peter Anthony Greenwood HOSKING, Auckland (NZ); Miriam Cather SIMPSON, Auckland (NZ)

(21) Appl. No.: 18/340,468

(22) Filed: Jun. 23, 2023

Related U.S. Application Data
(63) Continuation of application No. PCT/NZ2021/050230, filed on Dec. 23, 2021.
(60) Provisional application No. 63/130,328, filed on Dec. 23, 2020.

(51) Int. Cl. G01N 15/14

(52) U.S. Cl. CPC G01N 15/1434; G01N 2015/149; G01N 2015/0065

(57) ABSTRACT

In some embodiments there is provided a computer-implemented method for sorting cells. The method comprises receiving data comprising measurement datapoints for a plurality of cells, selecting a subset of the measurement datapoints using a region-of-interest, classifying the selected subset of measurement datapoints, and sorting the plurality of cells based on the classifying. In some embodiments, the method further comprises clustering previously received measurement datapoints and updating the region-of-interest based on the clustering by adjusting thresholds for the measurement datapoints.

### 1. TECHNICAL FIELD

The present disclosure relates to the classification of particles for downstream processes such as sorting. In particular embodiments, the particles comprise cells such as sperm cells.

### 2. BACKGROUND

The classification of particles having different characteristics is useful for many subsequent processes. For example, the classification of sperm cells into X and Y populations allows for downstream separation of sorting of these two populations. One category of sperm cells may be more desirable for certain types of animal farming. For example, bovine X sperm cells are preferred for the insemination of cows to produce predominantly female offspring for milking populations.

A major challenge in sex sorting of bovine sperm cells is the ability to achieve efficient discrimination between the X and Y populations.

In this specification where reference has been made to patent specifications, other external documents, or other sources of information, this is generally for the purpose of providing a context for discussing the features of the inventions disclosed herein. Unless specifically stated otherwise, reference to such external documents is not to be construed as an admission that such documents, or such sources of information, in any jurisdiction, are prior art, or form part of the common general knowledge in the art.

### 3. SUMMARY OF THE INVENTION

In some embodiments, there is provided a computer-implemented method for sorting cells. The method comprises determining measurement datapoints for a plurality of cells, selecting the measurement datapoints using a region-of-interest, classifying the selected measurement datapoints, and sorting the cells dependent on their respective classification. The region-of-interest or the classifying is automatically adjusted depending on previous measurement datapoints.

Embodiments may provide improved accuracy of cell classification, improved sorting efficiency, and/or adaptability to changing conditions or environment.

In some embodiments, there is provided a computer-implemented method for processing cells. The method comprises determining measurement datapoints for a plurality of cells, selecting the measurement datapoints using a region-of-interest, classifying the selected measurement datapoints into at least two populations according to a predetermined characteristic, and calculating a performance metric by comparing a number of datapoints in each population.

In some embodiments, there is provided a cell sorting apparatus. The cell sorting apparatus comprises a processor and memory configured to determine measurement datapoints for a plurality of cells, select the measurement datapoints using a region-of-Interest, classify the selected measurement datapoints, and sort the cells dependent on their respective classification. The region-of-interest or the classifying is automatically adjusted depending on previous measurement datapoints.

In some embodiments, there is provided a cell processing apparatus. The cell processing apparatus comprises a processor and memory configured to determine measurement datapoints for a plurality of cells, select the measurement datapoints using a region-of-interest, classify the selected measurement datapoints into at least two populations according to a predetermined characteristic, and calculate a performance metric by comparing a number of datapoints in each population.

Aspects of the inventions may also be said broadly to consist in the parts, elements and features referred to or indicated in the specification of application, individually or collectively, in any or all combinations of two or more of said parts, elements or features, and where specific integers are mentioned herein that have known equivalents in the art to which a said invention relates, such known equivalents are deemed to be incorporated herein as if individually set forth.

### 4. BRIEF DESCRIPTION OF THE DRAWINGS

The invention will now be described by way of example only and with reference to the drawings in which:

FIG. 1 is a schematic diagram of a system for sorting cells according to some embodiments;

FIG. 2 is a plot illustrating the classification of measurement datapoints corresponding to two fluorescent pulse integral measurement channels according to known approaches;

FIG. 3 is a schematic diagram of a system for classifying cells according to some embodiments;

FIG. 4 is a plot illustrating the classification of measurement datapoints corresponding to two fluorescent pulse integral measurement channels according to some embodiments;

FIG. 5 is a flow chart illustrating a method of detecting, classifying and sorting cells according to embodiments;

FIG. 6 illustrates the clustering of cell measurement datapoints for assessing orientation and discrimination of cell type;

FIG. 7 is a flow chart illustrating a method of determining an orientation efficiency metric; and

FIG. 8 is a flow chart illustrating a method of training a classifier.

FIG. 9 illustrates experimental results comparing a previously known classification approach (left) with an approach (right) according to some embodiments.

### 5. DETAILED DESCRIPTION OF THE INVENTION

In the claims, as well as in the specification above, all transitional phrases such as “comprising,” “including,” “carrying,” “having,” “containing,” “involving,” “holding,” “composed of,” and the like are to be understood to be open-ended, I.e., to mean including but not limited to. Only the transitional phrases “consisting of” and “consisting essentially of” shall be closed or semi-closed transitional phrases, respectively.

The term “about” as used herein means a reasonable amount of deviation of the modified term such that the end result is not significantly changed. For example, when applied to a value, the term should be construed as including a deviation of +/−5% of the value.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Whenever a range is given in the specification, for example, a temperature range, a time range, or a composition range, all intermediate ranges and subranges, as well as all individual values included in the ranges given are intended to be included in the disclosure.

The following sets forth specific details, such as particular embodiments or examples for purposes of explanation and not limitation. It will be appreciated by one skilled in the art that other examples may be employed apart from these specific details. In some instances, detailed descriptions of well-known methods, nodes, interfaces, circuits, and devices are omitted so as not obscure the description with unnecessary detail. Those skilled in the art will appreciate that the functions described may be implemented in one or more nodes using hardware circuitry (e.g., analog and/or discrete logic gates interconnected to perform a specialized function, ASICs, PLAs, etc.) and/or using software programs and data in conjunction with one or more digital microprocessors or general purpose computers. Nodes that communicate using the air interface also have suitable radio communications circuitry. Moreover, where appropriate the technology can additionally be considered to be embodied entirely within any form of computer-readable memory, such as solid-state memory, magnetic disk, or optical disk containing an appropriate set of computer instructions that would cause a processor to carry out the techniques described herein.

Hardware implementation may include or encompass, without limitation, digital signal processor (DSP) hardware, a reduced instruction set processor, hardware (e.g., digital or analogue) circuitry including but not limited to application specific integrated circuit(s) (ASIC) and/or field programmable gate array(s) (FPGA(s)), and (where appropriate) state machines capable of performing such functions. Memory may be employed to storing temporary variables, holding and transfer of data between processes, non-volatile configuration settings, standard messaging formats and the like. Any suitable form of volatile memory and non-volatile storage may be employed including Random Access Memory (RAM) implemented as Metal Oxide Semiconductors (MOS) or Integrated Circuits (IC), and storage implemented as hard disk drives and flash memory.

Some or all of the described apparatus or functionality may be instantiated in cloud environments such as Docker, Kubenetes or Spark. This cloud functionality may be instantiated in the network edge, apparatus edge, in the local premises or on a remote server coupled via a network such as 4G or 5G. Alternatively, this functionality may be implemented in dedicated hardware.

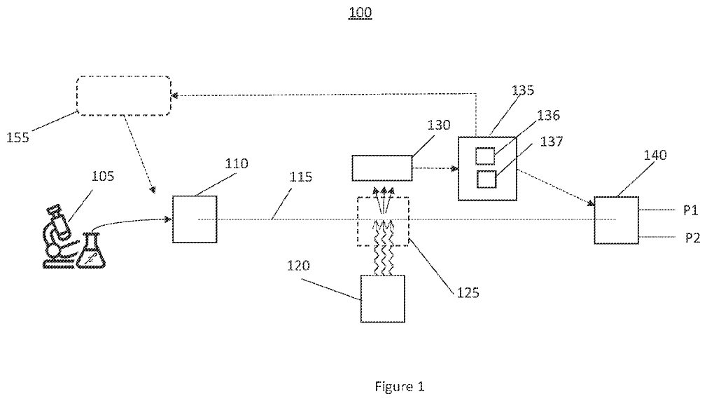

FIG. 1 illustrates a sorting system 100 comprising a preparation station 105 which delivers prepared cells to an input arrangement 110 which delivers the cells into a flow 115 for downstream processing. The flow of cells 115 may be a laminar flow carried within a microfluidic channel for example. One or more illuminators 120, for example an ultraviolet (UV) illuminator or other irradiation devices, irradiate the cells at an inspection station 125. Irradiation of the sperms cells causes them to emit illumination patterns such as scattered or fluorescent light which is detected by one or more detectors 130. Measured characteristics of the detected illumination patterns generate one or more signals which are forwarded to an analysis unit 135.

The analysis unit 135 comprises a processor 136 and memory 137 and is configured to interpret these signals in order to control a sorting arrangement 140 which sorts the cells into different populations P1 and P2 depending on analysis of signals associated with those cells. For example, if the analysis unit 135 determines that a sperm cell has an X (or Y) chromosome classification based on analysis of its respective measurement signals, the sorting arrangement 140 is controlled to sort this cell into one population (e.g. P1). Everything else in the original flow 115 is sorted into the other population P2. In an example, separated X sperm cells may be collected for further use. The other population of cells may be discarded. In alternative embodiments, the sorting arrangement may sort cells classified as having a preferred first classification A versus a second undesired classification B, wherein the sorting arrangement (also known as a sorting apparatus) comprises irradiating the cells identified as having a first, second classification, or being unclassified. Said irradiated cells irradiated with a laser. Said irradiated cells may be moved due to radiation pressure (for example from a laser), or they may be ablated to result in loss of function, cell kill or damage to the undesired population of cells, thereby producing a population of cells enriched in the desired characteristic.

The preparation station 105 may comprise apparatus for staining batches of cells, for example sperm cells collected from a bull. Various other preparatory steps may be undertaken such as diluting a semen sample batch from the bull.

The input arrangement 110 accepts a particle flow containing a solution, for example an aqueous solution, of cells from the prepared batch, and may also accept a sheath flow which may also comprise an aqueous solution. In one example, the input arrangement 110 combines the particle flow and sheath flow to generate a controlled laminar flow containing the cells. The flow rate of the particle and sheath flows may be controlled, and the input arrangement may contain components arranged to control the particle and sheath flows in order to orientate and/or confine the cells within the laminar flow. As some cells are asymmetrical, orientating them in a preferred plane improves their interaction with downstream apparatus such as the detector 130 and the sorting apparatus 140. Furthermore, confining the cells within a narrow flow path improves the likelihood that the downstream illuminators 120 will be incident upon them as intended.

The effectiveness of the input arrangement 110 to orient and/or confine the cells may improve the efficiency of the overall sorting system 100. An example of an input arrangement for improving cell orientation and/or confinement is a delivery tube as described in International Patent publication WO2020/013903 which is incorporated herein by reference. Other arrangements may alternatively be used, for the input arrangement may be part of a cytometer.

The laminar flow of cells 115 generated by the input arrangement 110 may be delivered into the transportation tube 115 which may comprise a silicon or glass capillary having a microfluidic lumen for carrying the laminar flow with the oriented and confined cells.

In some embodiments, two UV illuminators 120 may be oriented to deliver perpendicular irradiation to each cell passing through the inspection area 125. Two or more detectors 130 may also be oriented perpendicular to each other in order to capture responsive fluorescent light emanating from different directions from the irradiated cells. The detectors may be photomultiplier tubes at 90 degrees to each other, and to the direction of the transportation tube 115. The UV illuminators 120 may be arranged to cause pulses of responsive fluorescent light. This is measured by the detectors resulting in two signals or channels of pulse measurements which are proportional to the intensity or power of the received fluorescent light and which correspond to perpendicular directions.

The measurement of fluorescent light from a cell may be affected by a number of factors including the orientation and confinement of the cell within the inspection area 125, the level of retained staining of the cell at the inspection area 125, as well as biological factors such as whether the cell is dead or abnormal. For sperm cells the measurement of fluorescent light also depends on the identity of the sperm sex chromosome (X or Y).

Given that the difference in measurement signal due to the X or Y classification is approximately only 3%, these other factors make accurate and efficient classification of sperm cells challenging.

The signals from the detectors may each correspond to a rapid “strike” applied to each cell to cause the detected signal. A pulse integral signal may be derived for each cell by integrating the individual responsive fluorescent emission pulses associated with an individual cell over a predetermined period. These pulse integral signals for each perpendicular direction or channel may be generated at the detector apparatus 130 or at the analysis unit 135.

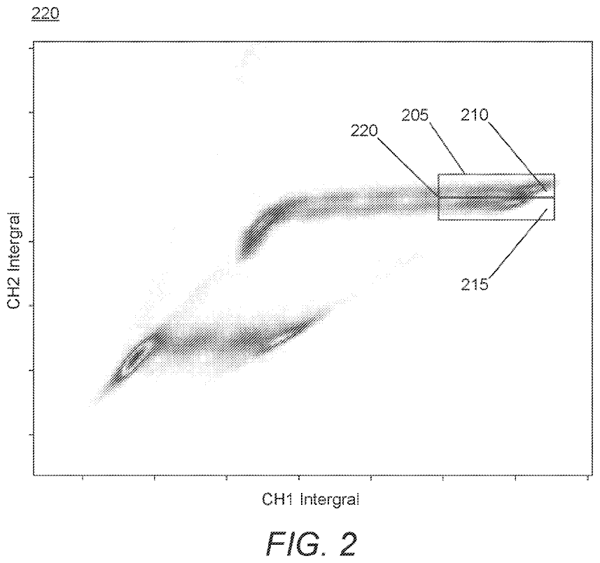

Together, measurements from two channels (eg channel 1 and channel 2) corresponding to different orientations of the cell represent a measurement datapoint. This may include for example a pulse integral of measurements taken from perpendicular directions. A plot of channel 1 (eg 0 degrees) and channel 2 (eg 90 degrees) measurement datapoints is shown in FIG. 2. Each datapoint represents a fluorescent pulse integral level measured in two perpendicular directions.

The analysis unit 135 of FIG. 1 analyses these signals or measurement datapoints to determine whether a cell should be classified as having a first characteristic A (for example part of the X population) (e.g. P1), and if so, controls the sorting arrangement 140 to sort the cell, for example by moving the cell into a different flow path. This allows all other cells, whether characteristic B (e.g. Y chromosome) or unclassifiable to remain in the original flow path so that they may be discarded. In an alternative embodiment, the Y-type sperm cells may be sorted by moving the cell into a different flow path. In a further embodiment, a sorting arrangement may be used that ablates a cell characterised as having (or lacking) a first characteristic in order to damage, kill or degrade the function of the cell. The unablated cells are allowed to pass through into one or more collection chambers. Some characteristic A-type cells may not be classified as such because they are non-viable (dead or abnormal), insufficiently stained upon reaching the inspection area 125, or poorly oriented or confined with respect to the measurement apparatus 120, 130. Staining is typically applied to cells in a vessel, however as the cells from the vessel reach the inspection area at different times the staining at the inspection area may vary from cell to cell. Improving the proportion of cells correctly classified as having a preferred characteristic A (e.g. X-bearing sperm cells) improves the efficiency of the sorting apparatus.

In an example, the sorting arrangement 140 may be a laser that is controlled to irradiate cells classified as having a first characteristic into one population P1 to cause them to move under radiation pressure into a different flow path. Everything else in the original flow path 115 is sorted into the other population P2 and remains in this flow path. Alternatively, everything identified as having a second characteristic B, or everything other than the wanted population (e.g. X sperm cells) may be moved into the different flow path. The two flow paths may be separated using a bifurcated microfluidic channel separator which splits the incoming microfluidic channel into two or more separate downstream channels. The separated cells may then be directed via their downstream channel to a collector where they are collected for further use. The other population of cells output of the other downstream channel may be discarded or used for other purposes. Such an arrangement is also described in International Patent publication WO2020/013903. However other sorting arrangements are possible, for example a sorting arrangement which electrostatically charges droplets containing cells according to their classification, the electrically charged droplets being guided into respective collectors by controlling an electric field.

The sorting system 100 may optionally comprise a monitor 155 which receives inputs from the analyser unit 135. The analyser unit may for example determine various operational metrics associated with the sorting system. Such metrics may include the rate of classified/sorted cells with a preferred characteristic (e.g. X sperm cells) compared with an expected number over a period or compared with a larger population or total number of cells—a sorting efficiency metric. The monitor 155 may be configured to issue an indicator such as a warning or alarm if these metrics fall outside a pre-determined range, for example above or below a defined threshold. In one embodiment, the indicator indicates that the region-of-interest (ROI) and/or a classifier separator need to be recalibrated. In another embodiment, the indicator issued by the monitor 155 may alternatively or additionally send a signal to modify the operation of the sorting system 100, for example to shut it down to prevent further wasting of the prepared cells.

In a further alternative, the indicator issued by the monitor may adjust some operational parameter of the sorting system such as adding more stain to remaining cells before delivery to the delivery tube arrangement, changing the flow rates of the sheath and/or particle flows, adjusting the geometry or interaction of components within the input arrangement 110 which may then change orientation and/or confinement parameters of the cells in the laminar flow.

FIG. 2 is a plot of measurement datapoints with respective channel 1 and channel 2 Integral levels. The plot is a 2D histogram of many measurement datapoints collected over a measurement period. The concentrations of datapoints in the lower left of the plot represent non-viable cells (dead or deformed), poorly stained cells, poorly oriented or confined cells. The Y-shaped concentration of datapoints in the centre and upper right of the plot represent viable cells that may be adequately stained and reasonably well oriented and confined. The upper horizontal arm of this Y-shaped concentration may correspond to a higher likelihood of one classification of cell (eg X sperm cells), and the lower horizontal arm may correspond to a higher likelihood of another classification of cell (eg Y sperm cells). The base region of the Y-shaped concentration towards the left in the plot corresponds to viable cells that are too difficult to classify, for example because they are poorly oriented. The upper and lower arms merge toward the base and it is increasingly difficult to distinguish between the two types of cell.

Classification of cells which differ in fluorescent intensity can be achieved by focusing on the right-hand portion of the Y-shaped concentration where it is easier to distinguish the two types. A region-of-interest (ROI) 205 is manually selected and a linear separator 220 within the ROI is also manually selected. Thereafter, subsequent cells may be classified as one type (eg X) if datapoints within the ROI have Ch1 and Ch2 values which intersect on one side of the separator 220—these datapoints are indicated by region 210. Subsequent cells may be classified as another type (eg Y) if datapoints within the ROI 205 have Ch1 and Ch2 values which intersect on the other side of the separator 220—indicated by region 215. It can be seen that only a small proportion of datapoints can be classified and therefore sorted, wasting a large number of cells of interest. This low efficiency increases the cost and time associated with producing cell samples enriched with a preferred characteristic e.g. X sperm cells.

It is also observed that the datapoints corresponding to viable cells may change over time, even within a single production run. For example, staining may degrade over time resulting in artefacts such as the Y-shaped concentration of datapoints moving within the plot, such as moving higher up the Ch2 axis. As the ROI 205 and separator 220 are set manually, this may result in datapoints within the ROI 205 being incorrectly classified. It may also result in fewer datapoints intersecting within the ROI 205. This phenomenon further reduces sorting efficiency and accuracy. This issue may be handled by manually resetting the ROI 205 and separator 220, however this requires significant operator input.

In some embodiments the analysis unit 135 uses a machine learning model to classify the cells which improves the classification efficiency and accuracy compared with known approaches such as using a linear separator to classify X and Y populations. The machine learning model may adapt over time, for example by continuing training using production data. This may improve classification accuracy and accommodate changes in the position of the Y-shaped concentration of datapoints over time.

In some embodiments the analysis unit 135 uses clustering of datapoints to determine a region-of-interest (ROI). The clustering may be used to focus the ROI and may also adapt to changes in the position of the Y-shaped concentration of datapoints over time.

In some embodiments, the use of a machine learning based classifier and clustering to focus the ROI may be combined to improve classification (and sorting) accuracy and efficiency. This may be further improved by employing adaptive techniques for the classifier and/or clustering.

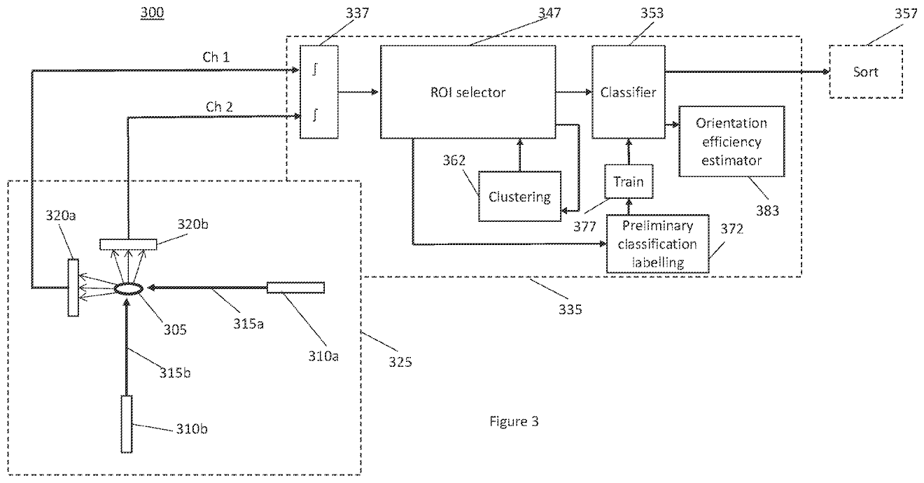

FIG. 3 illustrates part of a sorting system 300 comprising a measurement apparatus 325 and an analysis unit 335 according to embodiments. The measurement apparatus 325 comprises two UV illuminators 310a, 310b oriented perpendicular to each other and to a direction of travel of cells 305 through a transportation tube (not shown). Opposing each UV illuminator is a corresponding detector 320a, 320b each oriented perpendicular to each other. Each UV illuminator 310a, 310b directs UV light at passing cells which respond by emitting fluorescent light which is detected by the detectors 320a, 320b. The output from each detector 320a, 320b represent two measurement channels Ch1, Ch2 which are input to the analysis unit. In other arrangements, the UV illuminators and/or detectors may be oriented at different angles to each other. There may also be additional UV illuminators and/or detectors. Where UV illuminators are referred to herein, it will be appreciated by those of skill in the art that alternative illumination devices may alternatively be employed such as different wavelength light beams, lasers, X-rays, Infrared, microwave.

Alternatives to measuring responsive fluorescence may include measuring polarisation or scattering of laser beams or other electromagnetic radiation incident on cells, as well as the absorption and emission of different wavelengths of electromagnetic radiation such as Infrared.

The analysis unit 335 comprises an integrator function 337 coupled to the Ch1 and Ch2 inputs, a region-of-interest or ROI selector function 347 coupled to the integrator, and a classifier function 353 coupled to the ROI selector. The analyser unit or function uses the Ch1 and Ch2 inputs to classify the cell 305 associated with them. The classification output may be used to control a sort function 357, such as switching on a laser to apply radiation pressure to a cell, or controlling an electric field for sorting of cells according to flow cytometry methods. The analysis unit may also comprise one or more of a clustering function 362, an initial classification labelling function 372, a training function 377 and an orientation efficiency estimator function 383. The various functions may be implemented by suitably programmed computer or signal processing hardware, analogue or digital circuitry.

The integrator 337 integrates signal pulses received in response to the detectors measuring fluorescent light and may be integrated over a period corresponding to one or more illuminator pulses and the passing of a cell in front of the illuminators 310a, 310b. The pulse integrals for each channel Ch1, Ch2 associated with a cell represent a measurement datapoint.

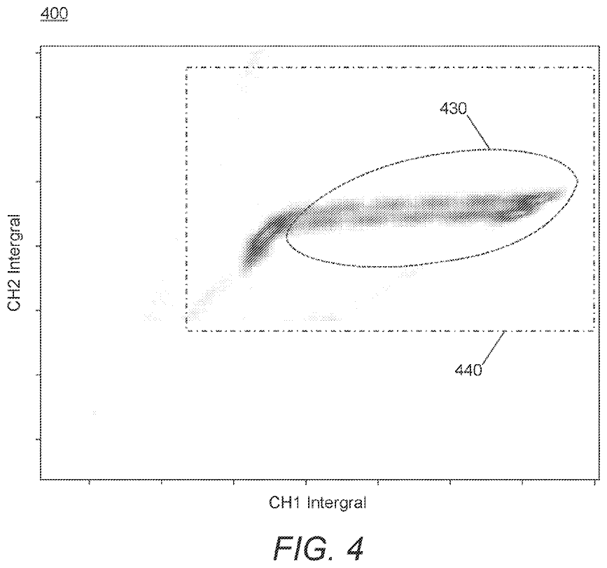

FIG. 4 is a 2D histogram showing measurement datapoints corresponding to Ch1 and Ch2 integral values. This is similar to FIG. 2 but has an adaptive ROI 440. Region-of-interest (ROI) 440 corresponds to viable cells and may initially be set to known ranges of Ch1 and Ch2 integral values based on experimental measurements. The ROI may then adapt over time using clustering as described below. In another example, the ROI may initially be set using pattern recognition to detect the characteristic y-shaped pattern shown and to centre the ROI around this. Region 430 represents the cells that may be classified using some embodiments. This compares favourably with the region of cells 205 in FIG. 2 that may be classified using previously known approaches. The region 430 corresponds to well oriented, viable and well stained cells that are suitable for classification by embodiments. This region is considerably larger than the ROI 205 of FIG. 2.

Returning to FIG. 3, the measurement datapoints are passed from the integrator function 337 to the ROI selector function 347 which removes some of these datapoints from consideration, for example because they represent unviable, poorly stained or poorly oriented cells. This corresponds to removing measurement datapoints outside the ROI 440 of FIG. 4. Viable but outlier datapoints may also be removed where the ROI selector uses clustering. For example, an initial ROI 440 may be selected using experimentally derived thresholds wide enough to encompass the Y-shaped pattern of viable and oriented cells whose location may vary across the Ch1 and Ch2 axes depending on external factors such as staining and the source animal. A more focussed ROI 430 may then be determined using clustering which eliminates outliers and thereby improves the classification accuracy The ROI selector 347 may be implemented using Ch1 and Ch2 threshold values corresponding to the focussed ROI 430.

The ROI selector may utilise clustering to remove datapoints distant from a main concentration. Continuous clustering also enables adaptation to changes in the incoming datapoints, for example due to changes in staining of the cells or a slight deviation in the flow path of the cells.

Examples of clustering algorithms that could be employed include K-means and K-medoids; mini-batch K-means and K-medoids; gaussian mixture modelling (GMM), balanced iterative reducing and clustering using hierarchies (BIRCH); density-based spatial clustering of applications with noise (DBSCAN); affinity propagation; agglomerative clustering; mean shift; spectral clustering; and ordering points to identify the clustering structure (OPTICS).

The ROI or clustered measurement datapoints are then forwarded to the classifier function 353. The classification function classifies the incoming datapoints into classes with a particular characteristic A or B, for example corresponding to X and Y sperm cells. Various classification models may be employed for example a trained machine learning model such as a neural network, a support vector machine or a hyperplane generated using machine learning. These classification models may be pre-trained then used in production mode, or the production mode itself may be used to further train the models to improve and/or adapt classification to changing input, for example due to changing staining of inspected cells. Different models may be trained for different circumstances, for example for use with different stains, different bulls or other animal species.

The classifier function 353 outputs a classification decision for each received datapoint which corresponds to classifying the cells as having characteristic A or B, for example sperm calls as X or Y. The classification of cells as characteristic A e.g. X sperm cells, may then be used to initiate some action such as a sort action 357. For example, this output may be used to control a laser to irradiate cells to cause them to move into a different flow path, or to ablate them.

These embodiments may facilitate improved classification and hence sorting accuracy resulting in a collection with a higher percentage of cells with a desired characteristic, for example X sperm cells. Efficiency may also be improved as a greater number of cells can be classified compared with known approaches.

The ROI selector function 347 may be adapted to continuously cluster production datapoints and adjust the focussed ROI thresholds accordingly. This means that the focussed ROI may follow changes in incoming datapoints, due for example to changes in stain retention over time. This may cause the concentration of data points to move, however using periodic or continuous clustering automatically centres the filter around the concentration which enables classification to continue unhindered. This compares with manual approaches which require operator monitoring and manual ROI adjustments.

The classifier function 353 may alternatively or additionally be adapted by ongoing training using production datapoints. For example, a historical collection of production datapoints may be employed as training data or additional training data for a machine learning model employed as the classifier. The training datapoints may be clustered and separated into two clusters. Separate clusters of datapoints may be initially delimited using a polynomial or spline function, with datapoints on one side of the function labelled as one class (eg X) and those falling on the other side of the separator function being labelled as a different class (eg Y). This corresponds with preliminary or approximate classification labelling function 372. These labelled datapoints may then be used to train or further train the classifier model using supervised learning, for example at train function 377. By applying recent production datapoints as top-up training data, the classification model may be continually improved and/or adjusted to accommodate changes in the environment such as reduced stain retention or cells from different animals affecting the datapoints.

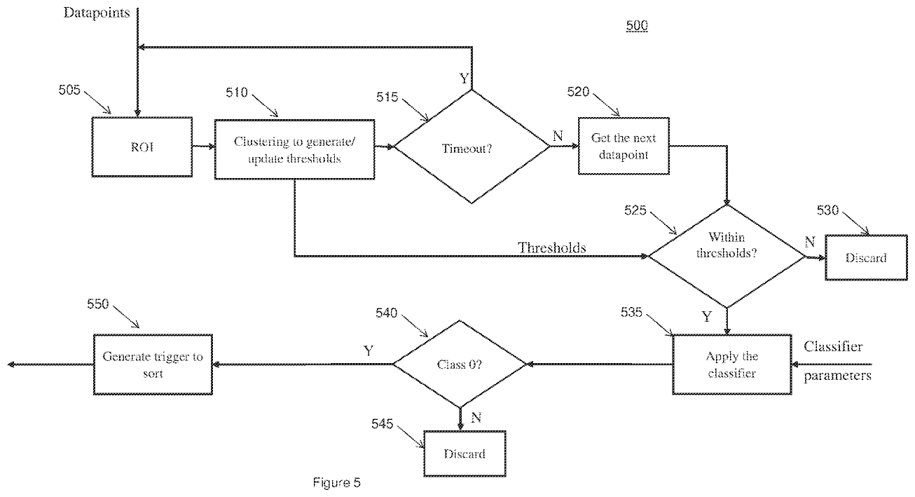

FIG. 5 illustrates a method of classifying datapoints corresponding to cells. This may be implemented by the analyser unit of FIG. 3 or by alternative hardware and/or software. The method 500 receives datapoints, for example pulse integrals from at least two channels corresponding measurement values in orthogonal directions.

At 505, the method filters the datapoints using an ROI filter. The ROI may initially correspond to pre-set or pattern recognition derived thresholds 440 of FIG. 4.

At 510, the method clusters the initially filtered datapoints, for example using any of the clustering algorithms previously described. The clustering is used to determine thresholds for more focussed filtering or the more focussed ROI 430 as shown in FIG. 4. These thresholds or cluster boundaries are then forwarded to 525.

At 515, the method determines whether a timeout period has expired and if not (N), the current datapoint is passed forward at 520. If the timeout period has expired (Y), the method loops back to 505 to start a new period for clustering received datapoints in order to update the focussed ROI thresholds.

At 525, the method determines whether the current measurement datapoint is within the focussed ROI thresholds most recently set by the clustering algorithm at 510. If the datapoint is outside the thresholds (N), the cell associated with the datapoint is marked for discarding. This state may be used to control a downstream sorting process, for example to control a laser to apply, or not apply radiation pressure to the cell so that it deviates from, or remains in, respectively a discard flow path. If the datapoint is within the thresholds (Y), the datapoint is passed for classification at 535.

At 535, the method classifies the datapoint as belonging to a cell with a characteristic A (eg class 0), or a cell with characteristic B (class 1). This may be implemented using any suitable classification, including for example: a non-linear separation hyperplane; a rules-based classifier (using fuzzy or non-fuzzy logic); a support vector machine (SVM); gaussian process classifier; a naïve bayes classifier; a neural network. These classifiers may be pre-trained then employed in a production setting to classify real or non-training datapoints. In some embodiments, the classifier parameters may be updated based on analysis of production datapoints, for example by continuously training a neural network or retraining an SVM to update a classification hyperplane. In another example, the ROI clustering may be used to adjust the location of the classification hyperplane. For example, if the median Ch2 value of the focussed ROI 430 is found to have increased by 5%, the Ch2 values of the classification hyperplane may be increased by 5%.

It has been experimentally observed that SVM classification can achieve 98% accuracy which significantly outmatches known manually configured separator approaches. Such accuracy improves the end product, for example sorted X sperm cell samples with a low proportion of Y sperm cells. This also reduces wastage of cells as more of the cells can be classified accurately.

At 540, the method determines the classification of the current datapoint, and associated cell, and if it has not been classified as being associated with a desirable cell (N), the cell is marked for discarding at 545. If the datapoint or cell is classified as being a desirable cell (Y), the method moves to 550.

At 550, the method controls a sorting arrangement e.g. a laser to apply radiation pressure to move the classified cell into a “collect” flow path. Sorting sperm cells using a laser is described for example in International Patent publications WO2014017929A1 and WO2020/013903 which are incorporated herein by reference. Other laser sorting arrangements may alternatively be used. The method may alternatively be used to control other sorting arrangements which do not rely on lasers, such as electrostatically charged droplet-based sperm cell sorting, or ablation of unwanted cells for example.

Returning to FIG. 3, the analyser unit 335 may also comprise an orientation efficiency estimation function 383 which analyses output from the classifier 353. The orientation efficiency may be used to provide sorting system monitoring for an operator and may also be used to control upstream processes such as shutting down the system if the orientation efficiency falls too low.

The orientation efficiency is a measure of the proportion of cells which are adequately oriented with respect to a reference direction. Sperm cells and other cells such as red blood cells are asymmetric having a flat oval shape, with perpendicular dimensions defining a face or large surface plane and a short dimension defining a thickness of an edge (as well as partially defining an orthogonal short surface plane). In some embodiments the sorting laser may be arranged to achieve optimal performance when its direction of propagation is perpendicular to the face or large surface plane. In this case, the reference direction is perpendicular to the laser propagation direction. The reference direction may alternatively correspond with the orientation of the characterising lasers 310a, 310b and/or detectors 320a, 320b. Asymmetric cells oriented within a certain range of the reference direction may still be sufficiently well oriented for laser-based sorting or other processes. Other cells which fall outside this range, for example asymmetric cells presenting their edge to the lasers, may result in measurement datapoints that cannot be classified and/or sub-optimal sorting.

A low orientation efficiency metric is an indication that the sorting system is configured sub-optimally resulting in the wastage of cells; for example because their fluorescence emissions cannot be accurately measured due to not being well oriented with respect to the illuminators or detectors. A low orientation metric may also indicate that even if accurately classified, many cells may not be properly sorted due to poor orientation with respect to a laser when this is used for sorting.

Referring to FIG. 4, cell orientation efficiency can be determined by comparing the number of datapoints within the focussed ROI 430 with the number of datapoints overall corresponding to all cell measurements, although different definitions could alternatively be used. This may be calculated by:

$$η = \frac{{Σ}_{A}}{{Σ}_{B}} * 100 \%$$

wherein Σ_{A} is the number of cells in the focussed region of interest 430, and Σ_{B} is the number of cells in another region which may correspond to all cell datapoints, or all viable cell datapoints. Whichever populations of cell datapoints are used, this metric represents the orientation efficiency over a period of time. Over different time periods, the orientation efficiency may vary. If the orientation efficiency falls below a threshold, for example 30%, this may trigger an alarm or other measure to prompt reconfiguration of the sorting system to be undertaken in order to improve the measured orientation efficiency.

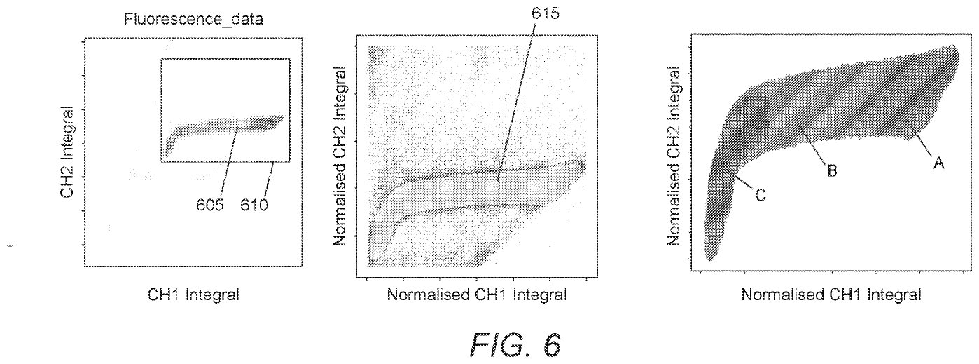

Alternative orientation efficiency metrics may be determined, for example as described below with respect to FIG. 6. Various other operational metrics may alternatively or additionally be determined. For example, changes in the filtering function thresholds may be monitored. If these exceed a certain level this may be indicative of a significant change to the cell sample being processed and may then initiate an operator warning or alarm, or the shutdown of the sorting system. Another example metric is comparing the number of cells classified as having a desirable characteristic A, e.g. X-bearing sperm cells, compared with the total number of cells.

In some embodiments, a machine learning based process (which, in some embodiments, is unsupervised) is provided which is configured to identify one or more dense populations of particles from a scatter plot of features extracted from fluorescent detection of particles in a partide/cell (e.g., sperm) sorting apparatus. In some embodiments, the size of the identified dense regions are then used to calculate particle orientation efficiency. Accordingly, and for example, particle orientation in a desired direction is, in some embodiments, a substantial factor for sorting the particle into a particular location/flow; thus, the ability to calculate particle orientation efficiency in this manner enables the comparison of alternative microchannel flowchips to identify configuration that promote better sorting functionality. In some embodiments, the identified dense regions are then used to identify sub-populations of particles. Accordingly, and for example, the identified members of the sub-populations are subsequently subjected to a sorting method.

Accordingly, in some of the disclosed embodiments: population density information, not directly visible to human eye, can be extracted using this approach; and unsupervised operation ensures robustness and avoids variations in the metric calculation arising from user interventions, especially when used to compare configurations.

Accordingly, in some embodiments, a method to identify one or more dense populations of particles from a scatter plot of features extracted from fluorescent detection of particles in a particle/cell (e.g., sperm) is provided and includes, fluorescing particles flowing in a microchannel of a sorting apparatus, extracting/imaging such fluorescence data, and determining a two-dimensional (2D) histogram of pulse shape features (e.g., Channel1 shape feature and Channel 2 shape feature) from the extracted fluorescence data. Channel 1 and Channel 2 shape features correspond to pulse shape features calculated from pulses from a detector placed at two different positions perpendicular relative to the flow respectively.

Referring to FIG. 4, a dense population of datapoints can be seen within region 440, some of which is included within the focused ROI 430. This dense population is hereafter referred to as an axial arm. Another dense population can be seen on the lower part of the figure, however this corresponds to unviable or poorly stained cells and is excluded from consideration using the initial ROI or mask threshold 440.

FIG. 6 illustrates a more detailed analysis of datapoints corresponding to cells with differing fluorescence, for example fluorescent integral measurements as previously described. The left plot illustrates an initial ROI 610 that may be set by a user to analyze the datapoints—this is the rectangle around the axial arm 605. The middle right plot shows the extracted axial arm datapoints 615, which may be extracted using a clustering technique. The right plot illustrates different regions within the axial arm which enables further analysis—a central lobe C, a middle arm B and a distal region A. These regions may be defined using a second clustering algorithm, for example grouping again based on density but with more granularity.

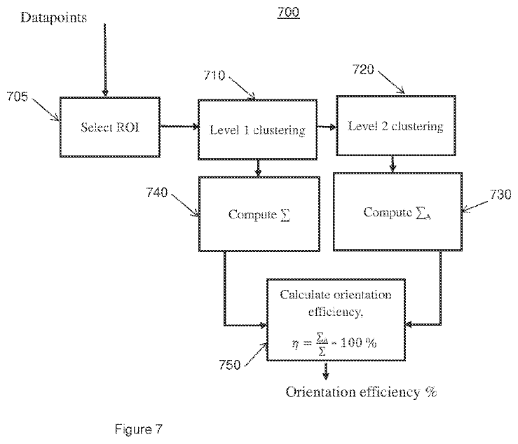

A method for determining an orientation efficiency metric according to some embodiments is illustrated in FIG. 7. This may be implemented to include at least one of, and in some embodiments, a plurality of, and in still further embodiments, all of the items illustrated.

At 705, the method prompts a user to specify a region of interest (ROI) 610, if required, in case there are regions of particle populations that should be avoided, to establish ROI data.

At 710, the method clusters the ROI data using (in some embodiments) an unsupervised clustering schema (e.g. a schema selected from the group consisting of K-means and K-medoids; mini-batch K-means and K-medoids; gaussian mixture modelling (GMM), balanced iterative reducing and clustering using hierarchies (BIRCH); density-based spatial clustering of applications with noise (DBSCAN); affinity propagation; agglomerative clustering; mean shift; spectral clustering; and ordering points to identify the clustering structure (OPTICS)) to separate out one or more dense regions of datapoints from sparse regions. This separates out the axial arm 615 of the 2D scatter plot as first clustered axial arm data (of interest) 615.

At 720, the method clusters the first clustered axial arm data via a second step of the unsupervised clustering schema (e.g. a schema selected from the group consisting of K-means and K-medoids; mini-batch K-means and K-medoids; gaussian mixture modelling (GMM), balanced iterative reducing and clustering using hierarchies (BIRCH); density-based spatial clustering of applications with noise (DBSCAN); affinity propagation; agglomerative clustering; mean shift; spectral clustering; and ordering points to identify the clustering structure (OPTICS)), with stricter parameter settings to identify dense/denser regions A, B, C within a dense region 615, establishing second clustered axial arm data A. Parameters which may be adjusted in a DBSCAN embodiment may include 1) epsilon: the distance between adjacent points can be reduced to find dense regions within dense regions and 2) minimum points: the minimum number of datapoints that must be present to tag as a cluster is reduced in order to find clusters within the first cluster.

At 730, the method calculates the number of points Σ_{A} in the dense oriented region A based on the second clustered axial arm data.

At 740, the method calculates one or more points Σ in the axial arm 615 based on the first clustered axial arm data (regions A, B and C).

At 750, the method calculates an orientation efficiency (e.g., particle orientation efficiency, using:

$$η = \frac{{Σ}_{A}}{Σ} * 100 \% .$$

The datapoints in the dense region A correspond to cells which are well oriented with respect to a reference direction. Well oriented cells are more likely to be well measured, classified and sorted, and therefore this particular orientation efficiency metric can be used to help improve the configuration of a cell sorting system. Cells in region C may be difficult to classify. Whilst cells in the middle region B may be adequately classified, increasing the proportion in region A further improves the system performance and efficiency.

Region A is where the most oriented cells are located and the comparison of regions A, B, C can be used to compare different upstream configurations such as flowchips and needles.

The regions A, B, C may be used to set a focused ROT for classification of datapoints. For example, the focused ROT may be set as region A, or region A and B. This approach may be used to set the focused ROT 430 of FIG. 4 in which a first clustering process is performed to separate an axial arm cluster 615 then a second clustering process is performed to identify well (A) and optionally reasonably well (B) dense population regions which are then used to set boundaries for the focused ROI 430.

As with the first clustering process, the second clustering process may also comprise any suitable algorithm, such as for example: K-means and K-medoids; mini-batch K-means and K-medoids; gaussian mixture modelling (GMM), balanced iterative reducing and clustering using hierarchies (BIRCH); density-based spatial clustering of applications with noise (DBSCAN); affinity propagation; agglomerative clustering; mean shift; spectral clustering; and ordering points to identify the clustering structure (OPTICS).

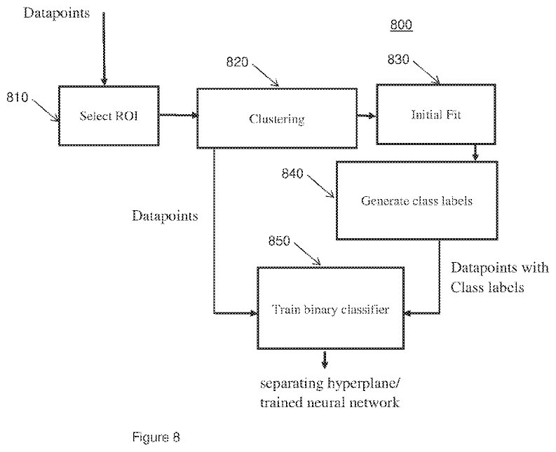

FIG. 8 illustrates a method 800 of continuous training of a classifier which may be employed in the analysis unit 335 of FIG. 3. A classifier such as 353 may be initially trained for use in a production setting or mode to classify and sort cells. The classifier may then be further trained using the production datapoints in order to update the classifier, for example to improve its accuracy or to adapt to changing conditions such as stain absorption or sperm cells from different bulls.

At 810, the method receives a plurality of datapoints and selects a region-of-interest (ROI). The datapoints may correspond to cell measurements and represent measurement datapoints for respective cells measured over a period. Examples are illustrated in FIGS. 4 and 6. The ROT selection may be implemented by manual setting by an operator, predetermined thresholds, pattern recognition or clustering as previously described.

At 820, the method clusters datapoints, for example using the level 1 clustering (710) of the method of FIG. 7 used in calculating an orientation efficiency.

At step 830, the method determines an initial line or function fit for the clustered datapoints. The initial fit may be achieved by polynomial fitting to define a line or smooth function corresponding to the datapoints. In one embodiment, a second order spline may be used.

At step 840, class labels (eg class 0 or class 1) are allocated to each datapoint depending on which side of the fitted polynomial they fall. into classes, for example class 0 corresponding to X sperm cells and class 1 corresponding to Y sperm cells.

At step 850, the datapoints from the clustering 820 are input into a classifier and the corresponding datapoints with labels are used for supervised training of the classifier.

In one example the classifier is an SVM and once trained, a separating hyperplane is obtained which then replaces the existing hyperplane used in the production classifier 353. Alternatively, an adjustment of the existing classifier hyperplane may be made depending on the difference between this and the newly generated hyperplane. The classifier may be updated or further trained periodically in this way.

In another example, the classifier is a neural network which is continuously trained using production datapoints and the corresponding labelled datapoints. This may be used for retraining an initial trained model provided at time of purchase but which may need to adapt to different conditions.

Some embodiments may provide one or more advantages. For example, the accuracy of cell classification and sorting is improved, and/or the efficiency of the sorting is improved by reducing wastage of desirable cells. This reduces costs and improves the final product. Automation may also be increased, reducing operator involvement and time.

Whilst some embodiments have been described with respect to a particular application, for example sex selection of bovine sperm, many other applications are possible. For example, sorting sperm from other farmed animals such as goats, sheep, deer, chickens and other poultry. Furthermore, in some embodiments, other types of cells may be sorted such as red blood cells and neurons.

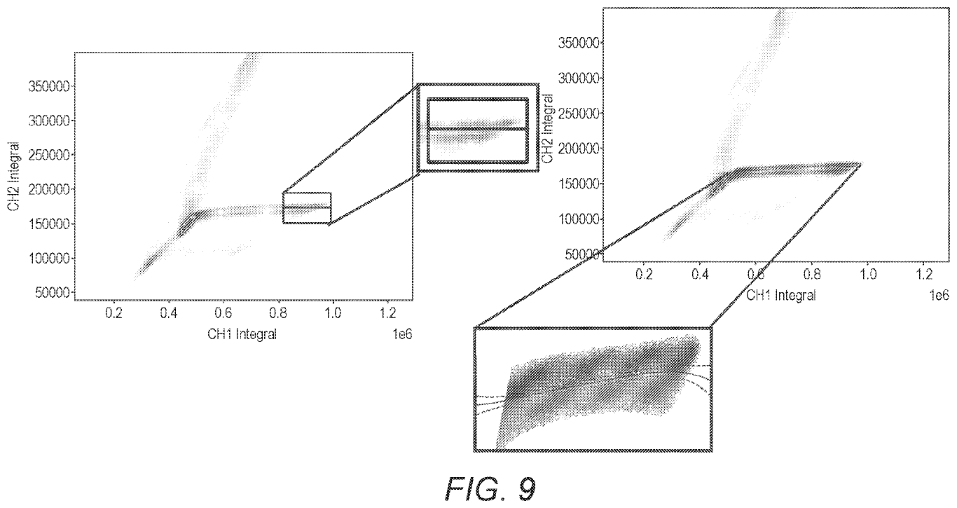

FIG. 9 illustrates experimental results comparing a previously known classification approach (left) with an approach (right) according to some embodiments. The 2D histogram on the left shows experimental results for sorting sperm cells into X and Y populations using a known manually configured linear separator approach. The Y-shaped pattern represents viable cells, and has an upper oriented arm corresponding to one population of cells (e.g. X sperm cells) and the lower oriented arm corresponding to another population of cells (e.g. Y sperm cells). A detail is shown of a region-of-interest (ROI). The ROI is relatively small, including only about one third of the cells in the two oriented arms. This is because a large tolerance must be associated with the ROI to accommodate variations in the measurement points over time and because of the coarseness of using a linear separator. It was found that the percentage of classified X cells as a percentage of all X cells in the oriented arms was approximately 55% and that the percentage of classified X cells as a percentage of all cells in the oriented arms was approximately 28%.

On the right, experimental results using an embodiment is shown which has a much larger ROI, effectively enabling use of 100% of the oriented arms. It was found that the percentage of classified X cells as a percentage of all X cells in the oriented arms was greater than 97.5% and that the percentage of classified X cells as a percentage of all cells in the oriented arms was greater than 49%. This represents a significant improvement in terms of classification and hence sort efficiency compared to known approaches. This effectively allows all or most oriented cells to be sorted which reduces wastage considerably and improves the concentration of wanted cells in the final collection.

Any and all references to publications or other documents, including but not limited to, patents, patent applications, articles, webpages, books, etc., presented anywhere in the present application, are herein incorporated by reference in their entirety.

As noted elsewhere, the disclosed inventive embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and apparatuses/devices which may further include any and all elements from any other disclosed methods, systems, and devices, including any and all elements corresponding to binding event determinative systems, devices and methods. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Also, some embodiments correspond to systems, devices and methods which specifically lack one and/or another element, structure, and/or steps (as applicable), as compared to teachings of the prior art, and therefore, represent patentable subject matter and are distinguishable therefrom (i.e., claims directed to such embodiments may contain one or more negative limitations to note the lack of one or more features prior art teachings).

Various inventive concepts disclosed herein may be embodied as one or more methods (as so noted). The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A computer-implemented method for sorting cells, comprising: receiving data comprising measurement datapoints for a plurality of cells;
selecting a subset of the measurement datapoints using a region-of-interest;
classifying the selected subset of measurement datapoints; and
sorting the plurality of cells based on the classifying.

2. The method of claim 1, wherein the region-of-interest is based on at least one of predetermined thresholds and pattern recognition.

3. The method of claim 1, further comprising
clustering previously received measurement datapoints; and
updating, based on the clustering, the region-of-interest by adjusting thresholds for the measurement datapoints.

4. The method of claim 3, wherein the clustering comprises one or more selected from the group consisting of: K-means and K-medoids; mini-batch K-means and K-medoids; gaussian mixture modelling (GMM), balanced iterative reducing and clustering using hierarchies (BIRCH); density-based spatial clustering of applications with noise (DBSCAN); affinity propagation; agglomerative clustering; mean shift; spectral clustering; and ordering points to identify the clustering structure (OPTICS).

5. The method of claim 1, wherein the classifying comprises using one or more selected from the group consisting of: a machine learning model; a non-linear function; a fuzzy classification; a gaussian mixture model (GMM); a statistical classifier; a convolutional and/or deep neural network; and a machine learning model trained using the previous measurement datapoints as labels.

6. (canceled)

7. The method of claim 5, wherein the classifying comprises applying the machine learning model trained using the previous measurement datapoints as labels, and wherein the previous measurement datapoints are initially classified using a non-linear function, the machine learning model being trained using the initially classified previous measurement datapoints.

8. The method of claim 1, wherein the classifying comprises applying a machine learning model to the selected subset of measurement datapoints, and wherein the machine learning model is one selected from the group consisting of: Kernel Support Vector Machine (k-SVM); deep and/or convolutional neural network; a Gaussian process classifier (GPC); rules-based classifier; and decision tree base classifier.

9. The method of claim 1, further comprising comparing a number of cells in a first population over a time period with a number of cells in a second population over the time period to calculate a sort efficiency parameter.

10. The method of claim 9, wherein the first population is one or more of a number of cells classified as having a predetermined characteristic and a number of cells in the region-of-interest, and wherein the second population is one or more the total number of cells and a number of cells in the region-of-interest.

11. The method of claim 9, further comprising performing an action when the sort efficiency parameter is below a threshold.

12. The method of claim 11, wherein the action is one or more of issuing a warning or alarm stopping the method for sorting cells, and performing an adjustment to an upstream cell delivery process.

13. The method of claim 1, wherein the plurality of cells are sperm cells and the measurement datapoints are derived from illumination pattern measurements from at least two different directions, at least some of the plurality of cells being classified according to a predetermined characteristic and wherein the sorting separates the classified cells from other cells.

14. The method of claim 13, wherein the measurement datapoints are fluorescent measurements from detectors oriented at an angle to each other, the cells being transported in a laminar flow and classified as cells having a characteristic A or B.

15.-27. (canceled)

28. A cell sorting apparatus, comprising a processor and memory configured to:
receive data comprising measurement datapoints for a plurality of cells;
select a subset of the measurement datapoints using a region-of-interest;
classify the selected subset of measurement datapoints; and
sort the plurality of cells; based on the classification.

29. (canceled)

30. The apparatus of claim 28, wherein the region-of-interest is based on one or more thresholds and the processor and the memory are further configured to:
cluster previously received measurement datapoints; and
update, based on the clustering, the region-of-interest by adjusting thresholds for the measurement datapoints.

31. The apparatus of claim 30, wherein the processor and the memory are configured to cluster the previously received measurement datapoints using one or more selected from the group consisting of: K-means and K-medoids; mini-batch K-means and K-medoids; gaussian mixture modelling (GMM), balanced iterative reducing and clustering using hierarchies (BIRCH); density-based spatial clustering of applications with noise (DBSCAN); affinity propagation; agglomerative clustering; mean shift; spectral clustering; and ordering points to identify the clustering structure (OPTICS).

32. The apparatus of claim 30, wherein the processor and the memory are configured to classify the selected measurement datapoints using one or more selected from the group consisting of: a machine learning model; a non-linear function; a fuzzy classification; a gaussian mixture model (GMM); a statistical classifier; a convolutional and/or deep neural network; and a machine learning model trained using the previous measurement datapoints as labels.

33. (canceled)

34. The apparatus of claim 32, wherein the processor and the memory are configured to classify the selected measurement datapoints using the machine learning model trained using the previous measurement datapoints as labels, and wherein the previous measurement datapoints are initially classified using a non-linear function, the machine learning model being trained using the initially classified previous measurement datapoints.

35-61. (canceled)

62. A processor-readable storage medium storing instructions that when executed on a processor cause the processor to perform a method according to claim 1.

63. (canceled)

64. The method of claim 1, wherein the classifying the selected measurement datapoints comprises:
applying a classifier to the selected measurement datapoints; and
updating the classifier based on previous measurement datapoints.
